(19) United States
(12) Patent Application Publication
(10) Pub. No.: US 2026/0368885 A1
(43) Pub. Date: Nov. 19, 2026

(54) DIGITAL IMAGING-BASED HAND, FOREARM AND MEDICAL COAT HYGIENE MONITORING METHOD AND SYSTEM

(71) Applicant: HANDINSCAN ZRT., Debrecen (HU)

(72) Inventors: Tamás Péter HAIDEGGER, Budapest (HU); Bence TAKÁCS, Pilisjászfalu (HU); Péter HEGYI, Budapest (HU); Péter Gábor SZERÉMY, Szeged (HU); Júlia BÁNSÁGHI SZÁVA, Budapest (HU); Péter RÓNA, Biatorbágy (HU)

(21) Appl. No.: 19/142,581

(22) PCT Filed: Dec. 12, 2023
(86) PCT No.: PCT/HU2023/050089

§ 371 Date: Jun. 23, 2025

(30) Foreign Application Priority Data
Dec. 21, 2022 (HU) P2200496

(51) Int. Cl. G06T 7/00; G06V 10/70; G06V 20/64; G06V 20/66; G06V 40/10; G16H 40/20

(52) U.S. Cl. CPC G06T 7/0012; G06V 10/70; G06V 20/64; G06V 20/66; G06V 40/107; G16H 40/20; G06T 2207/10016; G06T 2207/10024; G06T 2207/10028; G06T 2207/20081; G06T 2207/30196

(57) ABSTRACT

The system (100) for imaging and evaluation of a scanned object, in particular a user's hands, forearms and/or medical coats, regarding the quality of disinfection, comprises a unique, personal user identification number, marking date and location; a scanner unit (112); a cloud-based database (120) for storing and processing scanned image data; and a communication system for connecting the scanner unit (110) to the database (120). The scanner unit (112) comprises an open-bore imaging unit (112) employing a plurality of RGB/-D cameras for imaging, said imaging unit (110) being configured to record images or a video stream of a scanned object; a machine learning-based image processing unit (114); an illuminating unit for uniform illumination of an object to be scanned, at a predetermined narrow wavelength, and a user interface for presenting information about the image processing and analysis for the user and/or a healthcare institution.

### TECHNICAL FIELD

The invention relates to a method and a system for identifying appropriately disinfected hand surface areas, forearms, and coats of medical staff, based on digital image processing methods, and the method to compare and verify that to good infection prevention practices through improved hand hygiene quality compliance.

### BACKGROUND OF THE INVENTION

The unprecedented rapid spread of COVID-19 was also caused by inadequate personal hygiene practices (Khamis, Alaa, et al. “Robotics and intelligent systems against a pandemic.” Acta Polytechnica Hungarica 18.5 (2021 ): 13-35). According to the literature, 90-95% of non-professionals are not able to completely disinfect their hands, neither when washing hands with soap nor when using alcohol hand disinfection (Pittet, Didier “Improving compliance with hand hygiene in hospitals.” Infection Control & Hospital Epidemiology 21.6(2000 ): 381-386.). International literature has confirmed that 30% of healthcare workers routinely disinfect their hands improperly (Lehotsky, Ákos, et al., Orvosi Hetilap 158.29(2017 ): 1143-1148). In addition, more than 50% of them have some kind of pathogenic bacteria while providing care. At the same time, up to 50% of hospital infections could be prevented with proper hand hygiene, and this is among the top means of protection against pandemics. Beside hand surfaces, forearms are often overlooked sources of microbial contaminations (Journal of Hospital Infection Volume 75, Issue 2, June 2010, Pages 116-119). Coats of the medical staff may also be a vehicle to spread microbial contamination. (White Coats as a Vehicle for Bacterial Dissemination J. Clin Diagn. Res. 2012 October; 6(8): 1381-1384. Published online 2012 Oct. 10. doi: 10.7860/JCDR/2012/4286.2364 and Bacterial contamination of medical doctors' white coats as contributing factor to hospital acquired infections May 2017 International Journal of Biological and Chemical Sciences 11(1): 185 DOI: 10.4314/ijbcs.v11i1.15). To overcome this, however, a general change in behavior is requiredboth at home and in healthcare. A technical need to be solved is to carry out continuous, even multiple daily re-measurements and hand hygiene checks of healthcare workers and professionals.

The WHO (World Health Organization), the ECDC (European Center for Disease Prevention and Control), the American CDC (Centers for Disease Control and Prevention) and other professional organizations mention proper hygiene as a best practice against the pandemic. Thorough hand washing with soap and alcohol-based handrubs (ABHR) have proven to be effective against COVID-19 when applied properly, yet only if the complete coverage of the hand surfaces shall be attained.

Among the available solutions that can improve hand hygiene behavior, automated measurement, direct control and feedback proved to be the most effective (L. Szilágyi et al. BMC Infectious Diseases, 278, 2013). Thanks to the development of the hand hygiene scanners (T. Haidegger, Tamás Peter, et al. “Method for the disinfection and the quality control of the disinfection of the hands of a user and apparatus for carrying out the method.” U.S. Pat. No. 10,977,926. 13 Apr. 2021.), it is possible to routinely check the appropriate disinfection several times a day and operate the patient safety program as a Continuous Quality Improvement (CQI) & Total Quality Management (TQM) system at the institutional level, which is a general requirement imposed by serious professional organizations as WHO POPS, WHO ABRH Task Force and ISO (International Standardization Organization) 23447: 2023 standard.

The present invention provides a system and an associated method to deliver evidence-based verification of proper hand disinfection as a significant innovation in the hand hygiene market.

The system for imaging and evaluation of a scanned object, in particular a user's hands, forearms and/or medical coats, regarding the quality of disinfection, comprises-a unique, personal user identification number, marking date and location,
a scanner unit comprising:
   an open-bore imaging unit employing a plurality of RGB/-D cameras for imaging, said imaging unit being configured to record images or a video stream of a scanned object;
   a machine learning-based image processing unit;
   an illuminating unit for uniform illumination of an object to be scanned, at a predetermined narrow wavelength, and
   a user interface for presenting information about the image processing and analysis for the user and/or a healthcare institution.
a cloud-based database for storing and processing scanned image data;
a communication system for connecting the scanner unit to the database.

The method of imaging and evaluation of a scanned object, in particular a user's hands, forearms and/or medical coats, regarding the quality of disinfection, comprises the following steps carried out by the above system:
pre-processing images of the scanned object;
based on the images of the scanned object, segmentation and feature extraction;
based on the segmentation and feature extraction results, performing machine learning-based analysis;
performing region-based feature extraction on the basis of the machine learning-based analysis;
generating a 3D model for the object;
based on the 3D model of the object, computing the size of the scanned object;
based on the size of the scanned object, performing behavior and compliance analysis with regard to the user; and
presenting the graphical and numerical results of the scanning and analyzing processes to the user or to a personnel of a healthcare institution.

Preferred embodiments of the system and the method of the inventions are defined by the dependent claims.

### BRIEF DESCRIPTION OF THE DRAWINGS

FIG. 1 depicts a basic layout of the system for hand disinfection according to the invention.

FIG. 2 illustrates an optimized sensor arrangement for open-bore imaging in the system of the invention.

FIG. 3 shows the image processing workflow of the method according to the invention.

### DETAILED DESCRIPTION

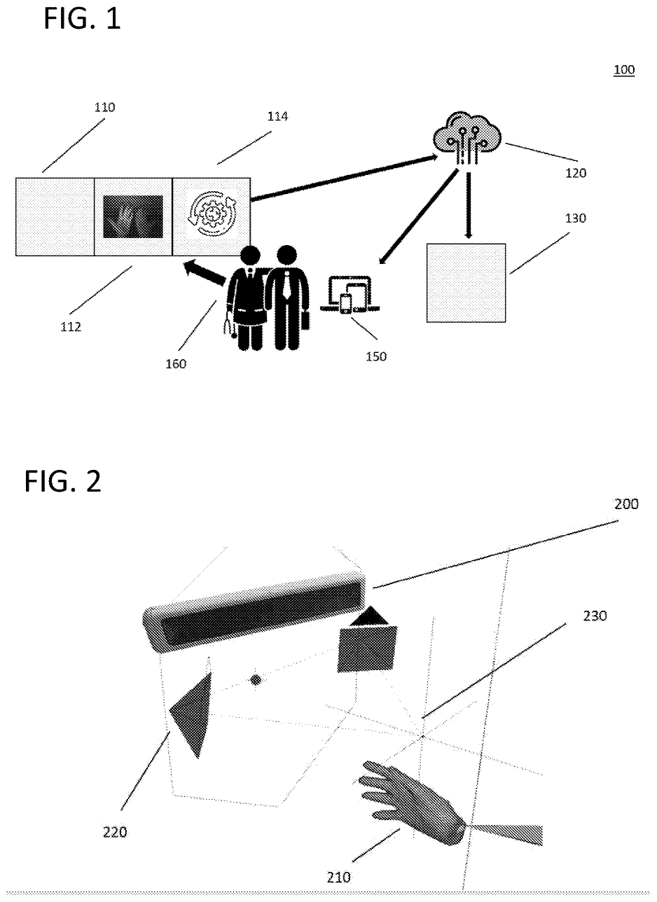

As shown in FIG. 1, the system 100 for hand, forearm and medical coat disinfection monitoring, in accordance with the present invention, consists of a scanner unit 110 including an evaluation software, connected with a local/cloud-based database 120 through a communication system. The scanner unit 110 comprises an imaging unit 112 and a machine learning-based image processing unit 114.

The database 120 is connected to an institutional information system 130, like a hospital information system, and personal mobile devices 150 of users 160 using the system 100 for disinfection purposes.

The scanner unit 110 of the system 100 according to the invention differs from the prior art scanners in that it comprises a multi-camera imaging unit using UV-A spectrum illumination, which works with lower light intensity and lower contrasts with respect to the conventional scanners. For hand disinfection, a riboflavin-based biocide marker or an Indocyanide Green (ICG) marker may be used.

As shown in FIG. 2, the spatially arranged camera system 200 of the multi-camera imaging unit provides the possibility of three-dimensional representation of a scanned object 210 or a scanned spatial area of an object, together referred to as “scanned object” in the present specification. Covering the required field of view 220 requires the selection or development of optical units with a deep depth of field and sufficiently low distortion. In order to produce a raw image of the same quality, uniform internal lighting is applied within the scanner unit, so that the position of the scanned object 210, for example a hand or a forearm, within the 3D reconstruction area 230 does not affect the lighting and the measured values. The recorded images made in parallel by the multiple cameras of the camera system 200 are combined into a single 3D reconstruction of the scanned object 210, which employs an innovative hardware-software solution.

The multi-camera system 200 consists of a plurality of optical units, where the light sources are emitting in the narrow wavelength range (Riboflavin: ca. 410 to 480 nm, ICG: ca. 750 to 850 nm), wherein optical filters with specific filter characteristics are installed in front of the light source and the cameras, and a wide-angle optics placed in front of the cameras produces selective imaging.

Preferably, as shown in FIG. 2, the layout of the cameras is optimized in order to create an image of the surface of the object 210, for example a hand, with as much coverage as possible. The great advantage of the marking agent selected is that it is not necessary to exclude either natural or other artificial light sources during imaging, thus the size of the scanner unit 110 can be reduced.

The multi-camera system 200 makes it possible to produce higher-resolution raw images from several angles, thereby achieving better signal/noise quality even from areas that are not in the optimal zone of imaging. The spatially arranged camera system 200 also provides 3D representation through spatial reconstruction. Covering the field of view makes the selection of optical units with a deep depth of field and sufficiently low distortion.

A multi-core, special purpose CPU/GPU central communication unit and interfaces may be integrated in the scanner unit 110 for communication with external devices and systems. Further key elements include the simultaneous data stream reading of the parallelized camera system and the synchronization of the image processing carried out by the multiple cameras, which are based on a proprietary internal data protocol and adjacent controllers.

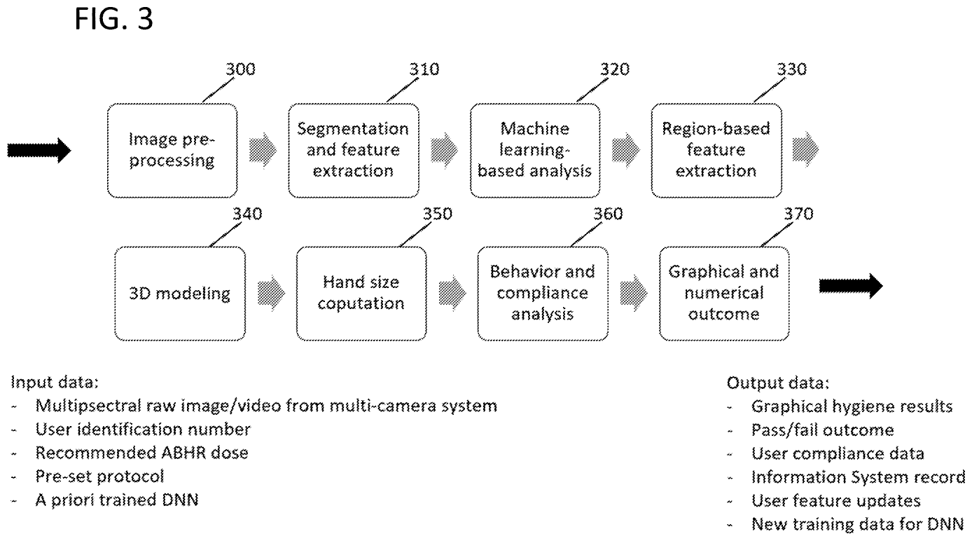

FIG. 3 illustrates the image processing workflow. Before starting the image processing workflow, the user completes the disinfection process according to a predefined protocol, including the application of an ABHR (or more generally, an alcoholic training solution) marked with a specific tracer. After the user's performing hand disinfection, he or she exposes their hands, forearms and/or the medical coat to the camera system for 3D scanning.

In step 300, image pre-processing is used to interpret a data series that may change dynamically in time, the image data generated by the multiple cameras. In step 310 the critical information (position and configuration of the human hand, arm and medical coat) is segmented in the given image field, either working based on still image or video feed as input data. In step 310, features of the scanned object are also extracted. In step 320, based on the extracted features of the scanned object, the camera images are processed and analyzed through machine learning algorithms, employing a combination of convolutional and modified, pre-trained U-NET networks (deep neural networks, DNNs) (Ronneberger et al. 2015, October. U-net: Convolutional networks for biomedical image segmentation. In Intl. Conf. on Medical image computing and computer-assisted intervention (pp. 234-241). Springer, Cham). The implemented learning algorithm may further employ synthetically generated data structures. In addition, the speed and reliability of the image processing system are guaranteed in accordance with professional user expectations, via hardware-accelerated Artificial Intelligence (Al) methods and with a number of implemented data analysis and audit support functions.

The system is capable of fast, multi-angle synchronized Monocrom NIR/RGB/RGB-D imaging in a fast operation mode, due to the custom-developed multi-camera system. The machine learning-based segmentation algorithm (step 310) enables the production of higher-resolution raw hand images even from several angles, thereby achieving better signal quality even from areas that have been difficult to detect so far. The image processing method of the invention is based on the latest Al-based deep neural networks (DNN) (Badrinarayanan et al. IEEE Transactions on pattern analysis and machine intelligence, 39(12), 2481-2495, 2017.),

The second layer of DNN-based image processing unit provides specific evaluation on the segmented image data in step 330, in which region-based feature extraction is carried out. In this step specific information on the intensity of each pixel, attributed to proper hand rubbing technique, the presence of ring and bracelets, as artifacts, the objective size of the hand and quality of the overall hand disinfection are determined. The region-based feature extraction process of step 330 links the ill-performance on specific hand segments to gaps in the knowledge of hand hygiene. The results may be stored in the cloud-based or local database coupled to the scanner unit, and may be reported back to the user through any kind of communication device (such as a personal mobile device) or through the user interface of the scanner unit.

The employed technical methods take into account the latest UI/UX scientific results in the field of behavior change. In addition, there is a serious scientific potential in the systematically collected data, the database created from the measurements could be greatly expanded through the development, which could serve as a basis for further research, especially for tracing the omitted areas back to the individual movements, which can implement the WHO's 6-step protocol evidence-based reassessment on the hand and proper forearm disinfection and coat hygiene.

Instead of a passive database, the system of the invention applies an online reporting system, enhanced into an interactive communication interface, where the user can not only retrieve the data, but can also change the method of data collection and communicate with the individual units.

The location, exact time and outcome of each measurement may be matched against the user's individual profile, expected behavior, working pattern and overall compliance with the quality management system defined by the health-care institution.

Based on the extracted region-based features of the scanned object, in step 340, a 3D model of the object is generated by the scanner unit, and using the 3D model, the hand size is computed in step 350. Then in step 360, the scanner unit performs behavior and compliance analysis with regard to the user, and finally, in step 370, the graphical and numerical results of the scanning and analyzing processes are presented to the user or to the personnel of the healthcare institution.

1. A system (100) for imaging and evaluation of a scanned object, in particular a user's hands, forearms and/or medical coats, regarding the quality of disinfection, the system comprising:
a unique, personal user identification number, marking date and location,
a scanner unit (112) comprising:
an open-bore imaging unit (112) employing a plurality of RGB/-D cameras for imaging, said imaging unit (110) being configured to record images or a video stream of a scanned object;
a machine learning-based image processing unit (114);
an illuminating unit for uniform illumination of an object to be scanned, at a predetermined narrow wavelength, and
a user interface for presenting information about the image processing and analysis for the user and/or a healthcare institution.
a cloud-based database (120) for storing and processing scanned image data;
a communication system for connecting the scanner unit (110) to the database (120).

2. The system according to claim 1, wherein the image processing unit (114) is configured to identify a user′ hand, forearm and/or coat on the basis of the processed and analyzed images or video stream.

3. The system according to claim 1, wherein the image processing unit (112) is configured to reconstruct a complete 3D surface of the user's hand or forearm as a 3D computer model.

4. The system according to claim 1, wherein the image processing unit (112) is configured to identify, within a scanned image of an object, any one of rings, jewels, bracelets, watches or other artifacts.

5. The system according to claim 1, wherein the image processing unit (114) is configured to determine the exact physical size of a user's hand or forearm.

6. The system according to claim 1, wherein the image processing unit (114) is configured to identify the hand hygiene performance along different regions of the hand of a user, and to link it with particular hand hygiene technique, such as the elements of the WHO's 6-step protocol.

7. The system as claimed in claim 1, wherein the scanner unit (110) is configured to transfer the results of the image processing and analysis to a cloud-based database (120), and to match said results against the expected location, time and quality requirements and profile of the user.

8. The system as claimed in claim 1 or 7, wherein the scanner unit (110) is configured to compute a specific evaluation score value on the basis of a user's previous and current disinfection quality performance, and to aggregate the score values of different measurements into a learning curve or skill curve defined by a plurality of measurement score values.

9. A method of imaging and evaluation of a scanned object, in particular a user's hands, forearms and/or medical coats, regarding the quality of disinfection, the method comprising the following steps carried out by the system of claim 1:
pre-processing (300) images of the scanned object;
based on the images of the scanned object, segmentation and feature extraction (310);
based on the segmentation and feature extraction results, performing machine learning-based analysis (320);
performing region-based feature extraction (330) on the basis of the machine learning-based analysis;
generating (340) a 3D model for the object;
based on the 3D model of the object, computing (350) the size of the scanned object;
based on the size of the scanned object, performing behavior and compliance analysis (360) with regard to the user; and
presenting (370) the graphical and numerical results of the scanning and analyzing processes to the user or to a personnel of a healthcare institution.
